(19) United States
(12) Patent Application Publication
(10) Pub. No.: US 2022/0096398 A1
(43) Pub. Date: Mar. 31, 2022

(54) COMPLEXES AND COMPOSITIONS COMPRISING PROBUCOL AND USES THEREOF

(71) Applicant: MACQUARIE UNIVERSITY, New South Wales (AU)

(72) Inventors: Alfonso GARCIA-BENNETT, New South Wales (AU); Michael Hui Ong LAU, New South Wales (AU); Kalpeshkumar Chandrakant GIRI, New South Wales (AU)

(21) Appl. No.: 17/427,035

(22) PCT Filed: Jan. 31, 2020
(86) PCT No.: PCT/AU2020/050062

§ 371 Date: Jul. 29, 2021

(30) Foreign Application Priority Data
Jan. 31, 2019 (AU) 2019900289

(51) Int. Cl. A61K 31/10; A61K 47/52; A61K 47/69; A61P 39/06

(52) U.S. Cl. CPC A61K 31/10; A61P 39/06; A61K 47/6923; A61K 47/52

(57) ABSTRACT

The present invention relates to complexes comprising probucol or derivatives thereof and mesoporous silica, methods for producing such complexes and uses thereof. The present invention also relates to uses of the complexes in the treatment of inflammation- and oxidation-related diseases and disorders.

### FIELD

The present invention relates to complexes comprising probucol or derivatives thereof and mesoporous silica, methods for producing such complexes and uses thereof.

### BACKGROUND

Oxidative stress, defined as the cellular production of reactive oxygen species (ROS) that overwhelms the host antioxidant defenses, is known to cause various cardiovascular, metabolic, and neurodegenerative diseases. For example, the overproduction of ROS is known to promote the development of atherosclerosis in cardiovascular diseases. Oxidative stress of the endothelial cells lining the microvascular network of the brain disrupts the integrity of the blood brain barrier, allowing for the infiltration of harmful substances that cause neurodegeneration.

Reactive oxygen species are known to cause oxidative damage to various biomolecules including proteins and DNA, tissue damage, cell death, and inflammation. It is known that oxidative stress and neuroinflammation are interrelated, and both play a key role in a range of neurodegenerative diseases, including Alzheimer's disease, epilepsy, multiple sclerosis, and Parkinson's disease.

Mitochondria are the major source of ROS production causing oxidative stress that may be implicated in various diseases. The electron transport chain, located on the inner mitochondrial membrane, generates the primary ROS superoxide (O₂⁻) from the partial reduction of oxygen. The dismutation of O₂⁻ by enzymes in the mitochondrial matrix generates hydrogen peroxide (H₂O₂). Both O₂⁻ and H₂O₂ are known to generate secondary, highly reactive ROS including the hydroxyl radical (OH), hypochlorous acid (HOCl) and peroxynitrite (ONOO₂⁻), each of which may cause oxidative damage to biomolecules.

The cyclooxygenase (COX) enzyme is activated to catalyze the conversion of arachidonic acid (AA) to prostaglandin (PG)G₂ and PGH₂, which are further metabolized by enzymes to form the family of prostaglandins. There are two isoforms of the COX enzyme; COX-1 is constitutively expressed for normal physiological function, and the inducible COX-2 enzyme activated during the inflammatory response. Both COX-1 and COX-2 enzymes are found intracellularly in brain endothelial cells, glial cells and neurons, and catalyze the formation of pro-inflammatory prostaglandins in neuroinflammatory diseases. The mitochondria and COX enzymes represent the key therapeutic targets in treatments of neuroinflammation and neurodegenerative diseases.

Probucol (drawn below) is a diphenolic compound and is known to prevent the Cu′-mediated oxidation of cholesterol in low-density lipoprotein (LDL). The compound has previously been used to lower cholesterol in order to prevent cardiovascular disease or treat conditions such as atherosclerotic lesions, diabetes mellitus and xanthoma. Probucol is also known for its anti-inflammatory and anti-oxidant effects. Although probucol has previously been used in the treatment of such indications, its unfavourable physical characteristics have limited its use. Probucol is a crystalline solid, highly lipophilic and has a limited solubility in water of about 2 to 5 ng/mL. The low solubility of probucol in aqueous solutions results in a low bioavailability, requires a high dosage to be administered and thus presents difficulties to formulators seeking to provide an efficient dose of the drug.

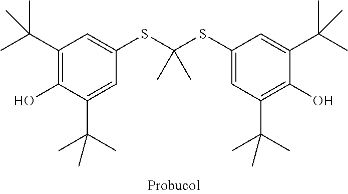 [SMILES: CC(C)(Sc1cc(C(C)(C)C)c(O)c(C(C)(C)C)c1)Sc1cc(C(C)(C)C)c(O)c(C(C)(C)C)c1]

Accordingly, improved formulations of probucol are desired where a sufficient dose can be delivered given its unfavourable solubility profile.

### SUMMARY OF THE INVENTION

According to a first aspect of the present invention, there is provided a complex comprising mesoporous silica and probucol or a derivative thereof, wherein at least a portion of the probucol or derivative thereof is present within the pores of the silica.

The probucol or derivative thereof present in the complex may be in an amorphous form. In an embodiment, the probucol or derivative thereof present within the pores of the silica may be in an amorphous form. In another embodiment, the probucol or derivative thereof present within the pores of the silica may be in an amorphous form, and probucol or derivative thereof in a crystalline form may be present on the exterior surface of the silica.

The probucol or derivative thereof present in the complex may be present in an amount up to about 60% by weight of the complex.

In an embodiment, the mesoporous silica may have an average pore size of between about 3 nm and about 30 nm. In an exemplary embodiment, the mesoporous silica may have a pore size of about 3.2 nm or 3.4 nm. In an exemplary embodiment, the mesoporous silica of the complex may have an average pore size of about 4 nm or 4.6 nm. In an exemplary embodiment, the mesoporous silica of the complex may have an average pore size of about 11 nm or 11.8 nm. In an exemplary embodiment the mesoporous silica may have a pore size distribution of between about 6 nm to about 20 nm.

The connectivity of the pores in the mesoporous silica may be two-dimensional (2D) or three-dimensional (3D). In an embodiment, the pores of the mesoporous silica may be two-dimensional. In another embodiment, the pores of the mesoporous silica may be three-dimensional.

According to a second aspect of the present invention, there is provided a pharmaceutical composition comprising a complex according to the first aspect and one or more pharmaceutically acceptable carriers, diluents or excipients.

According to a third aspect of the present invention, there is provided a method for preparing a complex according to the first aspect, the method comprising the steps of:
i) contacting mesoporous silica with a mixture of probucol in a solvent; and
ii) removing the solvent.

In an exemplary embodiment, the solvent is ethanol.

According to a fourth aspect of the present invention, there is provided a method for lowering cholesterol in a subject, the method comprising administering to the subject a complex according the first aspect or a composition according to the second aspect.

According to a fifth aspect of the present invention, there is provided a method for increasing the bioavailability of probucol in a subject, the method comprising administering to a subject in need thereof a complex according the first aspect or a composition according to the second aspect.

According to a sixth aspect of the present invention, there is provided a method for treating a cholesterol-related disease or disorder in a subject, the method comprising administering to the subject a complex according the first aspect or a composition according to the second aspect.

According to a seventh aspect of the present invention, there is provided a method for treating an inflammation- or oxidation-related disease or disorder in a subject, the method comprising administering to the subject a complex according to the first aspect or a composition according to the second aspect.

According to an eighth aspect of the present invention, there is provided a method for treating pain or inflammation in a subject, the method comprising administering to the subject in need thereof a complex according to the first aspect or a composition according to the second aspect.

According to a ninth aspect of the present invention, there is provided a method for inhibiting the activity of a cyclooxygenase enzyme in a subject, the method comprising administering to the subject in need thereof a complex according to the first aspect or a composition according to the second aspect.

According to a tenth aspect of the present invention, there is provided the use of a complex according to the first aspect in the manufacture of a medicament for lowering cholesterol.

According to an eleventh aspect of the present invention, there is provided the use of a complex according to the first aspect in the manufacture of a medicament for treating a cholesterol-related disease or disorder.

According to a twelfth aspect of the present invention, there is provided use of a complex according to the first aspect in the manufacture of a medicament for treating an inflammation- or oxidation-related disease or disorder.

According to a thirteenth aspect of the present invention, there is provided use of a complex according to the first aspect in the manufacture of a medicament for treating pain or inflammation.

According to a fourteenth aspect of the present invention, there is provided use of a complex according to the first aspect in the manufacture of a medicament for inhibiting the activity of a cyclooxygenase enzyme.

### BRIEF DESCRIPTION OF THE DRAWINGS

Embodiments of the invention are described herein, by way of non-limiting example only, with reference to the following figures.

FIG. 1. Plot of the amount of crystalline probucol (expressed as a percentage), as a function of the amount of probucol loaded into porous silica of different pore shapes (2D or 3D) and sizes (3.2 nm, 4.6 nm or 11.8 nm). The amount of crystalline material present in the silica increases if a drug loading of more than about 40% by weight of probucol is used.

FIG. 2. Plot of the surface area of the silica remaining after loading of probucol, as a function of probucol loaded into porous silica of different pore shapes (2D or 3D) and sizes (3.2 nm, 4.6 nm or 11.8 nm). The surface area remaining is at a minimum (i.e. the silica is filled) where the loading of probucol is between about 40% to 50% by weight for the types of silica tested.

FIG. 3. Plot of the amount of probucol released from porous silica loaded with probucol, as a function of the amount of probucol loaded into porous silica of different pore shapes (2D or 3D) and sizes (3.2 nm, 4.6 nm or 11.8 nm). The amount of probucol released from the silica is lower if silica with a smaller pore size (3.2 nm) is used. The amount of probucol released from the silica is also lower if the amount of probucol is greater than 30-40% by weight, as the probucol is likely present in its crystalline (rather than amorphous) form.

FIG. 4. Plot of the amount of probucol released (in mg, left or as a percentage of total probucol, right) from a capsule containing probucol loaded into porous silica with either a 2D pore (11.8 nm) or 3D pore (4.6 nm). Both plots show an increase in the amount of probucol released with an increase in the capsule dose.

FIG. 5. Plot of the percentage of human cerebral microvascular endothelial cells showing a positive ROS (reactive oxygen species) response over time after administration of hydrogen peroxide alone or with Vitamin C, probucol or silica (AMS-6) loaded with probucol. Administration of silica loaded with probucol gives a decrease in the reactive oxygen species detected, showing the antioxidant activity of probucol delivered by the silica.

FIG. 6. Physical and structural characterization of examples of suitable calcined mesoporous silica: (A) AMS-6; (B) MCM-41; and (C) SBA-15. Scanning electron microscopy images (i) show agglomerated spherical particles for AMS-6 and MCM-41, and rod type morphology for SBA-15. Pore size and porous properties derived from nitrogen adsorption data are shown for each sample.

FIGS. 7A-C. Thermogravimetric analysis curves for examples of suitable mesoporous silica (AMS-6, SBA-15 and MCM-41) loaded with varying amounts of probucol (loading of probucol given as a weight percentage) and resulting in different amorphous states of the drug compound, indicated by the different decomposition temperatures in comparison to probucol alone.

FIGS. 8A-C. Nitrogen adsorption-desorption isotherm curves for calcined and probucol-loaded samples of silica. Silica samples are AMS-6, MCM-41 or SBA-15, without probucol or loaded with probucol at the given percentage loading. Samples loaded with greater amounts of probucol show a lower adsorption of nitrogen. Samples without probucol show the highest adsorption of nitrogen. Only adsorption branch is shown.

FIGS. 9A-F. Plots of the percentage of probucol released into simulated intestine fluid from porous silica (AMS-6, SBA-15 or MCM-41) loaded with probucol (loading of probucol given as a percentage by weight) against time.

FIGS. 10A-F. Plots of the percentage of probucol released into simulated intestine fluid from a capsule (weight given in milligrams) containing porous silica (AMS-6, SBA-15 or MCM-14) loaded with probucol (loading of probucol given as a weight percentage).

FIG. 11. Plot of the plasma concentration-time curves of probucol and AMS-6 with probucol loaded at 34.8 wt % after oral administration via gavage in comparison to the corresponding amount of crystalline probucol.

FIG. 12. Percentage of cells (human brain endothelial cells) with oxidative stress after incubation with 1 μg/ml LPS with or without the test compounds for 24 hours. The percentage of cells with oxidative stress was lower at all doses when exposed to probucol (30%) released from AMS-6 compared to crystalline probucol. Columns, from left to right, represent: HBEC with media only; HBEC+AMS-6PB 30% 0.1 μM+1 μg/ml LPS; HBEC+AMS-6PB 30% 1.0 μM+1 μg/ml LPS; HBEC+AMS-6PB 30% 10.0 μM+1 μg/ml LPS; HBEC+PB 0.1 μM+1 μg/ml LPS; HBEC+PB 1.0 μM+1 μg/ml LPS; HBEC+PB 10.0 μM+1 μg/ml LPS; and HBEC+1 μg/ml LPS.

FIG. 13. Percentage of cells with oxidative stress in cells incubated with 1 μg/ml LPS followed by addition of test compounds at shorter treatment times of 2, 4 and 6 hours. The percentage of cells with oxidative stress was lower at all doses when exposed to probucol (30%) released from AMS-6 compared to crystalline probucol, showing an enhancement in free radical scavenging. For each time point (2 hr, 4 hr, 6 hr) columns, from left to right, represent: HBEC with media only; HBEC+AMS-6PB 30% 0.1 μM+1 μg/ml LPS; HBEC+AMS-6PB 30% 1.0 μM+1 μg/ml LPS; HBEC+AMS-6PB 30% 10.0 μM+1 μg/ml LPS; HBEC+PB 0.1 μM+1 μg/ml LPS; HBEC+PB 1.0 μM+1 μg/ml LPS; HBEC+PB 10.0 μM+1 μg/ml LPS; and HBEC+1 μg/ml LPS.

FIG. 14. Cellular viability of human brain endothelial cells incubated with 1 μg/ml LPS with or without the addition of test compounds. The release of probucol (30%) from AMS-6 increased cellular viability at all doses and time points compared to crystalline probucol. For each time point (2 hr, 4 hr, 6 hr, 24 hr) columns, from left to right, represent: HBEC with media only; HBEC+AMS-6PB 30% 0.1 μM+1 μg/ml LPS; HBEC+AMS-6PB 30% 1.0 μM+1 μg/ml LPS; HBEC+AMS-6PB 30% 10.0 μM+1 μg/ml LPS; HBEC+PB 0.1 μM+1 μg/ml LPS; HBEC+PB 1.0 μM+1 μg/ml LPS; HBEC+PB 10.0 μM+1 μg/ml LPS; and HBEC+1 μg/ml LPS.

FIG. 15. Total cyclooxygenase (COX) activity in human brain endothelial cells incubated with 1 μg/ml LPS with or without the test compounds AMS-6 probucol (30%), crystalline probucol, and the potent COX enzyme inhibitor, indomethacin (INDO). The release of probucol from AMS-6 reduced total COX enzyme activity at all doses after 24 hours incubation compared to crystalline probucol and indomethacin. In top graph (24 hour incubation time), columns, from left to right, represent: HBEC (media only); HBEC+AMS-6PB 30% 0.1 μM+1 μg/ml LPS; HBEC+AMS-6PB 30% 1.0 μM+1 μg/ml LPS; HBEC+AMS-6PB 30% 10 μM+1 μg/ml LPS; HBEC+PB 0.1 μM+1 μg/ml LPS; HBEC+PB 1.0 μM+1 μg/ml LPS; HBEC+PB 10 μM+1 μg/ml LPS; HBEC+INDO 0.1 μM+1 μg/ml LPS; HBEC+INDO 1.0 μM+1 μg/ml LPS; HBEC+INDO 10 μM+1 μg/ml LPS; and HBEC+1 μg/ml LPS. In bottom graph (2 to 6 hour incubation time), columns, from left to right, represent: HBEC (media only); HBEC+AMS-6PB 30% 1.0 μM+1 μg/ml LPS; HBEC+PB 1.0 μM+1 μg/ml LPS; HBEC+INDO 1.0 μM+1 μg/ml LPS; and HBEC+1 μg/ml LPS.

FIG. 16. Solubility of probucol (% PB released) over time from capsules containing Syloid with a pore size 20-30 nm (Syloid-PB28.5%) and capsules containing low mesopore size silica: AMS-6 with a pore size of approximately 4 nm (AMS6-PB28.4%) and SBA-15 with a pore size of approximately 11 nm (SBA15-PB29.9%).

### DETAILED DESCRIPTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the term “about,” is understood to refer to a range of numbers that a person of skill in the art would consider equivalent to the recited value in the context of achieving the same function or result.

The present invention relates to complexes of mesoporous silica and probucol or a derivative thereof, where at least a portion of the probucol is present in the pores of the silica. Accordingly, in one aspect the present invention provides a complex comprising mesoporous silica and probucol or a derivative thereof, wherein at least a portion of the probucol or derivative thereof is present within the pores of the silica. In the discussion hereinbelow referring to probucol, the skilled person will appreciate that this discussion not only encompasses probucol itself, but also derivatives thereof. Exemplary derivatives of probucol are described herein.

The term “complex” as used herein in relation to mesoporous silica and probucol means a product derived from the association of mesoporous silica and probucol, where probucol is located on one or more surfaces of the silica, and where at least a portion of the probucol located on a surface of a pore of the silica.

In producing a complex of the present invention, typically probucol is dissolved in a solvent and silica is added to the mixture. Subsequent removal of the solvent leads to the impregnation of the probucol within the pores of the silica. The low solubility of probucol is attributed to the crystalline form of the drug, however the present inventors have now found that loading probucol in its amorphous form (i.e. when dissolved in solvent) into pores of silica by contacting the silica with a solution of probucol allows for a portion of the probucol to be located within the pores of the silica in its amorphous form. This leads to a complex comprising mesoporous silica and probucol, where the probucol has a higher solubility in aqueous environments, and subsequently, an improved bioavailability of probucol. In addition to increasing the solubility of probucol, complexes of the present invention also lead to an increased half-life of probucol upon administration of the complexes. While impregnation of the mesoporous silica with a solution of probucol leads to the amorphous probucol in the pores of the silica, probucol in its crystalline form on the exterior surface of the silica may still be produced if the loading of probucol exceeds a certain amount.

Silica is also known as silicon dioxide and has the formula SiO₂. In accordance with embodiments of the present invention the silica may be amorphous and may have a particle size ranging from, for example, between about 50 nm and about 50 μm. Silica materials that are suitable for use in the complexes of the present invention contain pores, i.e. the silica is porous. Examples of silica that may be suitable for use include mesoporous silica materials such as SBA-15, SBA-16, MCM-41, AMS-6 or other surfactant-templated materials with pores larger than 3.4 nm and smaller than 30 nm. The skilled addressee will appreciate that the scope of the present invention is not limited by reference to any specific silicas, provided the silica possesses a pore size distribution in the range between 3.4 nm and 30 nm.

The type of silica selected to provide complexes of the present invention affects the amount of probucol that may be loaded into the pores, and subsequently, the rate of release of the probucol upon administration and contact with an aqueous environment. Specifically, the pore size of the silica affects the amount of probucol that may be loaded into the pores and also the rate at which the probucol is then released upon administration and contact with an aqueous environment. Without wishing to be bound by theory, the present inventors believe that using a different type of silica with a different pore size may lead to complexes of probucol with different loadings of probucol and subsequently, different release rates of probucol. The type of silica used may be selected in order to provide a complex with a specific loading of probucol or specific rate of release of probucol. The selection of the type of silica and the pore sizes of the silica for different applications and to achieve desired probucol loadings and release rates is within the skill and expertise of the skilled addressee and the selection may be made using ordinary skill in the art without undue experimentation or need for further invention.

Porous silica may have pores of different sizes, for example, silica may be microporous, mesoporous or macroporous. Exemplary silica pore size may be between about 1 nm to about 200 nm. In accordance with particular embodiments of the present invention, the silica is typical mesoporous silica, which is a silica with a pore size between about 3 nm and about 30 nm. In particular embodiments, the silica is mesoporous silica with a pore size between about 3 nm and about 20 nm. In particular embodiments, the silica is mesoporous silica with a pore size between about 3 nm and about 18 nm. In particular embodiments, the silica is mesoporous silica with a pore size between about 3.4 nm and about 18 nm. By way of example only, the mesoporous silica may have a pore size of about 3 nm, 3.2 nm, 3.4 nm, 3.6 nm, 3.8 nm, 4.0 nm, 4.2 nm, 4.4 nm, 4.6 nm, 4.8 nm, 5.0 nm, 5.2 nm, 5.4 nm, 5.6 nm, 5.8 nm, 6.0 nm, 6.5 nm, 7.0 nm, 7.5 nm, 8.0 nm, 8.5 nm, 9.0 nm, 9.5 nm, 10.0 nm, 10.5 nm, 11.0 nm, 11.2 nm, 11.4 nm, 11.6 nm, 11.8 nm, 12.0 nm, 12.5 nm, 13.0 nm, 13.5 nm, 14.0 nm, 14.5 nm, 15.0 nm, 15.5 nm, 16.0 nm, 16.5 nm, 17.0 nm, 17.5 nm, 18.0 nm, 19 mm, 20.0 nm, 21 nm, 22 nm, 23 nm, 24 nm, 25 nm, 26 nm, 27 nm, 28 nm, 29 nm or 30 nm. In an exemplary embodiment, the mesoporous silica may have a pore size of about 3.4 nm. In another exemplary embodiment, the mesoporous silica may have a pore size of about 4.6 nm. In another exemplary embodiment, the mesoporous silica may have a pore size of about 11.8 nm.

In other embodiments, the silica is mesoporous silica where the pore size distribution is between about 6 nm to about 20 nm. While not wishing to be bound by theory, the inventors suggest that a silica pore size in this range allows for minimal crystallization of probucol on the external surface of the silica.

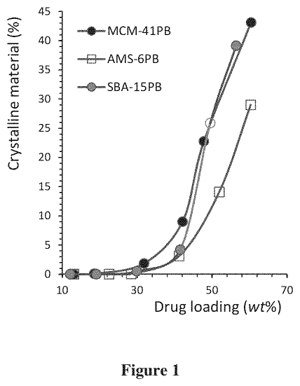

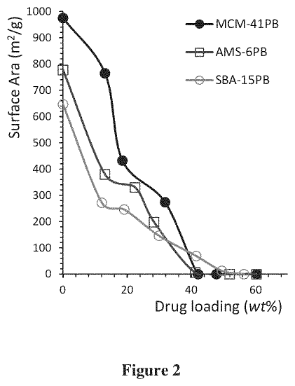

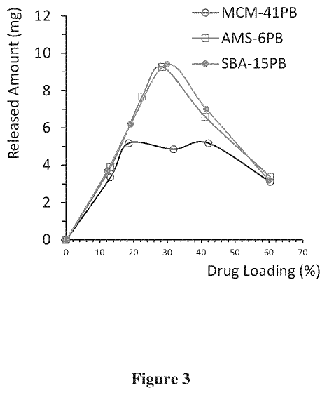

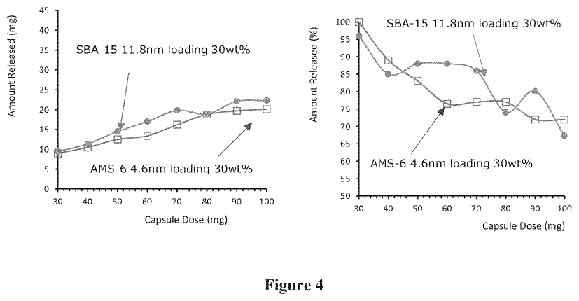

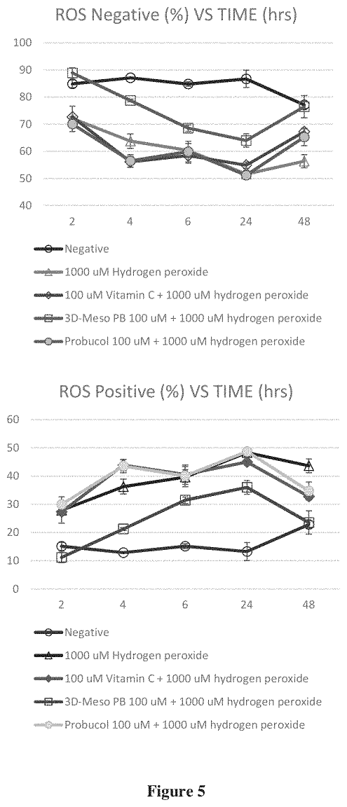

The present inventors have also found that the size of the pores in the silica also affects the amount of probucol that can be loaded and subsequently released. For example, a smaller pore size can result in an earlier onset of probucol crystallization, when loading probucol into the pores of the silica, which may be unfavourable since the crystalline form of probucol has lower solubility. Additionally, smaller pore sizes, such as about 3.4 nm, lead to a lower amount of probucol in its amorphous form being loaded into the pores of the silica. Where silica with a larger pore is used, such as about 11.8 nm, a greater amount of probucol can be loaded into the silica before the formation of crystalline probucol is observed. Increasing the loading of probucol in the pores of the silica also results in the accumulation of crystalline probucol on the exterior surface of the silica particle. This can be seen in FIG. 1, where the amount of crystalline material increases once the drug loading reaches an amount of about 40% by weight. Even where crystalline probucol is present on the exterior surface of the silica, the overall perceived solubility of probucol (as observed through the amount of probucol released upon contact with an aqueous environment) is increased due to the amorphous probucol present in the pores of the silica. FIG. 2 also suggests that once the loading of probucol reaches about 40% by weight, the pores of the silica are filled with probucol (as seen by a reduction in the surface area of the silica). An increase in the loading of probucol then leads to an increase in the amount of crystalline probucol that is likely located on the surface of the silica since the pores of the silica are full. The present inventors believe that the probucol loaded into the pores of the silica is retained in its amorphous form and that any additional probucol that is associated with the complex is present as probucol in its crystalline form. The amount of probucol released from such a complex is at a maximum when the loading of the probucol is about 40%. This can be seen in FIG. 3, suggesting that the released probucol is the amorphous probucol found in the pores of the silica, since an increased loading of probucol (attributed to the crystalline form of probucol) does not result in an increase in the actual amount of probucol released and detected.

The pores of the silica may be described as two-dimensional (2D) or three-dimensional (3D). A 2D pore may be described as having a honeycomb-like morphology, with channels forming through the silica to create pores. A 3D pore may be described as having an indefinite, sponge-like morphology that extends throughout the silica, where connectivity between the pores exists. The present inventors have found that the morphology of the pores in the silica affect the extent of probucol being loaded into the pores in its amorphous form and also the amount of probucol that is later released, with silica having a 3D pore network achieving greater release of probucol, when compared with silica having a 2D pore network.

The selection of the pore size of the silica and also the loading of probucol in the complex affects the rate at which the probucol is released and that for a given pore size, there is loading of probucol that results in an optimal release rate. For example, a smaller pore size (such as about 3.2 nm pore size for a 2D pore) results in release of less probucol, when compared to a larger pore size (such as about 11.8 nm). Without wishing to be bound by theory, the inventors believe that the desired rate of release of probucol from a complex may be governed by appropriately selecting both the pore type and the pore size of the silica. Such selection may be made by the skilled addressee using ordinary skill in the art without undue experimentation or need for further invention.

The probucol in a complex of the present invention may be present in an amount of, for example, up to about 60% by weight of the complex. In accordance with the present invention, at least the portion of the probucol present within the pores of the silica is present in the amorphous form. In an embodiment, a portion of the probucol in the complex and not residing in the pores of the silica may be in the crystalline form. The amount of probucol present in the complex of the present invention may be referred to as the loading of the probucol in the complex. In an embodiment, the loading of probucol in a complex of the present invention may be up to about 60% by weight of the complex. The loading of probucol in the complex may be about 5%, 10%, 15%, 20%, 25%, 30%, 35%, 40%, 45%, 50%, 55% or 60% by weight. In an embodiment, the probucol is present in an amount of about 30% by weight. In another embodiment, the probucol is present in an amount of about 40% by weight. The present optimal loading of the probucol that provides the highest rate of release of probucol. The present inventors have also found that at lower drug loadings, such as below about 20% by weight of probucol, the pores of the silica are only partially filled. When the loading of the silica is increased to about 40% by weight, the pores of the silica are substantially filled, and the surface area of the silica remaining is minimised. This can be seen in FIG. 2, where the surface area of the silica is at a minimum when the loading of probucol is greater than about 40% by weight. This is also confirmed in FIG. 3, where the amount of probucol released is a maximum at a loading of about 40% and any further increase in loading does not result in eventual release of the drug.

In another aspect of the present invention, there is provided a method for preparing a complex comprising mesoporous silica and probucol.

The complexes of the present invention may be produced by loading probucol (in any of its crystalline forms) into the pores of the amorphous silica. Probucol is typically first dissolved in a suitable solvent or mixture of solvents. Suitable solvents include C₁-C₆ alkanols, ketones, aliphatic hydrocarbons, aromatic hydrocarbons and mixtures thereof. Examples of solvents include, but are not limited to, methanol, cyclohexane, acetone, diethyl ether and mixtures thereof. A person skilled in the art would understand that the physical properties of a given solvent and its compatibility with probucol will govern the choice of solvent or solvents to be used. In an embodiment, the solvent is ethanol.

The amount of solvent required to dissolve the probucol, i.e. the ratio of probucol to solvent, may vary according to the nature of the solvent. A person skilled in the art would understand that the physical properties of the solvent, for example, the polarity of the solvent, will influence the amount of solvent required (and also the ratio of the probucol to the solvent required) to dissolve a given amount of probucol for the purposes of loading into the pores of the mesoporous silica. In an embodiment, probucol is dissolved in ethanol, where probucol and ethanol are present in an amount of about 1:5, 1:6, 1:7, 1:8, 1:9, 1:10, 1:11, 1:12, 1:13, 1:14 or 1:15 by weight. In a typical embodiment, probucol is dissolved in ethanol, where probucol and ethanol are present in an amount of about 1:10 by weight. The solubility of probucol in the solvent may be enhanced or assisted by sonication (i.e. the application of sound energy at ultrasonic frequencies) of the probucol-solvent mixture. In an embodiment, the mixture of probucol and ethanol is subject to a sonication step. In another embodiment, a mixture of probucol and ethanol in a ratio of about 1:10 by weight is subjected to a sonication step.

An amount of mesoporous silica is then added to the solvent containing the dissolved probucol. The mixture of silica and solution of probucol is allowed to stir for a time before the solvent is removed. In an embodiment, the mixture of silica and solution of probucol is stirred at room temperature for about 30 minutes. In an embodiment, the mixture is stirred at a rate of about 300 rpm. The solvent may be removed by known procedures, such as rotary evaporation under reduced pressure. In an embodiment, the solvent is removed by rotary evaporation under reduced pressure. In another embodiment, the mixture is heated to a temperature above room temperature while the solvent is removed by rotary evaporation under reduced pressure. In another embodiment, the mixture is heated to a temperature of about 40° C. while the solvent is removed by rotary evaporation under reduced pressure. The solvent may be removed by rotary evaporation, where the pressure is progressively decreased until the solvent is removed from the silica. In an embodiment, the solvent is removed under rotary evaporation at a pressure of about 800 mbar for about 10 minutes, followed by a pressure of about 100 mbar for about 20 minutes and then at a pressure of about 1 mbar for about 30 minutes. Removal of the solvent leads to a dried, powdered and amorphous silica with probucol impregnated in the pores of the silica. The present inventors have found that the probucol found within the pores of the silica is amorphous (as it is when dissolved in solvents), rather than crystalline, and that the amorphous nature of the probucol within the pores of the silica allows for greater solubility of the probucol when exposed to an aqueous environment. The dried silica may also comprise probucol on the exterior surface of the silica particles, which may be crystalline or amorphous, however the existence of crystalline probucol (i.e. the form of probucol with low aqueous solubility) on the silica surface still allows for the amorphous probucol contained with the pores of the silica to provide the enhanced solubility. The present inventors have found that the manner in which the solvent is removed, i.e. rate at which the solvent is removed under pressure, affects the formation of crystalline probucol on the surface of the silica. The pressure program described herein may contribute to minimizing the amount of crystalline probucol formed on the surface of the silica.

As described herein, complexes of the present invention may comprise probucol or a derivative of probucol. Suitable derivatives of probucol may include compounds where one or both of the phenolic hydrogens of probucol are replaced with another substituent. Examples of derivatives of probucol and procedures for their synthesis include the esters of probucol, as described, for example, in Canadian Patent No 2404943, in which the phenolic hydrogen is replaced with a carbonyl compound to provide an ester of probucol. Different esters may be provided if the substituent on the carbonyl compound is varied.

Without wishing to be bound by theory, the present inventors believe that even though the derivatives of probucol discussed here may have different physical characteristics and hence a different solubility profile to probucol itself, derivatives of probucol may also be incorporated into the pores of silica to provide a complex in a similar manner. A person skilled in the art would understand that owing to any differences in the physical properties of the derivatives of probucol, a different type of silica, pore shape and/or size may be necessary in order to provide a complex with the desired release properties. Other derivatives of probucol, i.e. non-ester derivatives, may also be included in complexes of the present invention.

The bioavailability of probucol depends upon the specific physical characteristics and solubility profiles of the molecule. Probucol complexed with mesoporous silica in accordance with the present invention displays greater solubility, and thus greater bioavailability in vivo, than uncomplexed probucol. As exemplified herein, the present inventors have shown that the time to achieve maximum concentration of probucol in vivo is reduced and the maximum concentration of probucol is increased, when a complex according to the present invention (compared to crystalline probucol) is administered. These results show that the complexes of the present invention have a different pharmacokinetic profile when compared to crystalline probucol alone. This suggests that the physical properties of probucol are different when a complex according to the present invention is prepared. Without wishing to be bound by theory, the present inventors believe that that probucol when complexed with mesoporous silica as described herein remains in its amorphous form, rather than its crystalline form (uncomplexed). This leads to the different physical properties of probucol that are now observed. Administration of a complex of probucol according to the present invention leads to increased solubility, and subsequently bioavailability, of probucol. Embodiments of the invention therefore provide methods for increasing the bioavailability of probucol when administered to a subject, when compared to the bioavailability of probucol in its crystalline, uncomplexed form. In some cases, the bioavailability of a complex of probucol or a derivative thereof, as described herein, may be between 3 to 10 times that of probucol in its crystalline, uncomplexed form.

The complexes of the present invention may be provided in the form of a composition, optionally with one or more pharmaceutically acceptable diluents, adjuvants and excipients. Accordingly, in one aspect of the invention there is provided a pharmaceutical composition comprising a complex described herein and one or more pharmaceutically acceptable carriers, diluents or excipients.

Compositions may be administered to subjects in need thereof via any convenient or suitable route such as by parenteral (including, for example, intraarterial, intravenous, intramuscular, subcutaneous), topical (including dermal, transdermal, subcutaneous, etc), oral, nasal, mucosal (including sublingual), or intracavitary routes. Thus compositions may be formulated in a variety of forms including solutions, suspensions, emulsions (including Pickering emulsions), and solid forms and are typically formulated so as to be suitable for the chosen route of administration, for example as an injectable formulations suitable for parenteral administration, capsules, tablets, caplets, elixirs for oral ingestion, in an aerosol form suitable for administration by inhalation (such as by intranasal inhalation or oral inhalation), or ointments, creams, gels, or lotions suitable for topical administration. The preferred route of administration will depend on a number of factors including the disease or disorder to be treated and the desired outcome.

The most advantageous route of administration for any given circumstance can be determined by those skilled in the art. For example, in circumstances where it is required that appropriate concentrations of the desired agent are delivered directly to the site in the body to be treated, administration may be regional rather than systemic. Regional administration provides the capability of delivering very high local concentrations of the desired agent to the required site and thus is suitable for achieving the desired therapeutic or preventative effect whilst avoiding exposure of other organs of the body to the compound and thereby potentially reducing side effects.

In general, suitable compositions may be prepared according to methods known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof. Pharmaceutical carriers for preparation of pharmaceutical compositions are well known in the art, as set out in textbooks such as Remington's Pharmaceutical Sciences, 20^{th} Edition, Williams & Wilkins, Pa., USA. The carrier will depend on the route of administration, and again the person skilled in the art will readily be able to determine the most suitable formulation for each particular case.

The compositions may be provided as a solid dosage form, optionally for oral administration. Such forms may include tablets, capsules, pills, powders and granules, where the complex is mixed with one or more pharmaceutically acceptable diluents, adjuvants and excipients. The solid compositions comprising complexes of the present invention may also be prepared with coatings and shells, such as enteric coatings and other coatings known the art. The distribution and release of the solid composition (and subsequently, the complex of the present invention) may be further modified, for example, the solid composition may be formulated to be a slow-release formulation or as part of a targeted delivery system. In addition to the pharmaceutically acceptable diluents, adjuvants and excipients that may be present in the composition, the compositions of the present invention may comprise a complex of silica and probucol and one or more other active agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propylparaben or sodium bisulphite. Suitable lubricants include magnesium stearate, stearic acid, sodium oleate, sodium chloride or talc. Suitable time delay agents include glyceryl monostearate or glyceryl distearate.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate. Polyoxyethylene sorbitan mono- or di-oleate, -stearate or -laurate and the like.

Emulsions for oral administration may further comprise one or more emulsifying agents. Suitable emulsifying agents include dispersing agents as exemplified above or natural gums such as guar gum, gum acacia or gum tragacanth.

The complexes and compositions of the present invention may be useful in the treatment and prevention of diseases and disorders where cholesterol levels are elevated. Due to its known solubility problems, the use of probucol has previously been limited, owing to the low bioavailability of the drug upon administration. Since the complexes of the present invention provide probucol in an amorphous form where the drug is more soluble in aqueous environments, and subsequently has a greater bioavailability, the complexes of the present invention and compositions comprising the complexes may be used in the treatment and prevention of cholesterol-related conditions. Additionally, since probucol is known to inhibit the Cu²⁺-mediated oxidation of low-density lipoprotein in cholesterol and also have other anti-inflammatory and anti-oxidant properties, the complexes of the present invention may also be used to treat or prevent diseases and disorders that are related to inflammation and oxidation.

Inflammation and pain may be mediated by cyclooxygenase (COX) enzymes that catalyse the formation of prostaglandins. Inhibiting the activity of cyclooxygenase can inhibit the oxidative pathways mediated by the enzymes and subsequently inhibit inflammation and pain. As exemplified herein, complexing probucol with mesoporous silica in accordance with the present invention significantly enhances the antioxidant properties of the amorphous probucol upon release from the pores of the silica, compared to uncomplexed crystalline probucol. Accordingly, embodiments of the present invention provide methods for the treatment of inflammation and pain.

In view of the enhanced antioxidant properties of amorphous probucol, the complexes and compositions described herein may be used for the treatment of a variety of diseases and conditions such as tissue injury, inflammatory disorders, pulmonary diseases, cardiovascular diseases, metabolic disorders, cancers and neurodegenerative disorders. Treatment with complexes of probucol may decrease the cellular damage caused by oxidative modification related to reactive oxygen species (ROS) that are generated in such pathologies. Administration of the complexes or compositions described may be used to mitigate the detrimental effects of ROS and associated cellular oxidative stress. Under conditions of oxidative stress, increased concentrations of ROS overwhelm cellular antioxidant defense mechanisms, causing oxidative damage to cells and the development of pathological conditions. Inhibition of COX enzymes may be related to the quenching of ROS and related species, such as superoxide, hydrogen peroxide, hydroxyl radicals, hypochlorous acid and peroxynitrite. Where COX enzymes and/or ROS and related species are implicated in a disease or disorder, inhibition of COX enzymes and subsequently quenching of ROS and related species may lead to inhibition of such diseases and disorders.

Examples of diseases and conditions that may be treated or prevented by administration of complexes or compositions described herein include metabolic diseases, cardiovascular diseases, cancers and tumours, inflammatory diseases, autoimmune diseases, neurological diseases, neurodegenerative diseases and other diseases and disorders associated with oxidative stress. Exemplary diseases and disorders include, but are not limited to, type 2 diabetes, insulin resistance, elevated cholesterol levels, nephropathies, myocardial infarction, progression of left ventricular dysfunction, remodeling in tachycardia-induced heart failure, atherosclerosis, heterozygous familial hypercholesterolemia, xanthoma regression, restenosis, lung metastasis of breast cancer, lung fibrosis, rheumatoid arthritis, stroke, ageing, neural and synaptic plasticity in brain ageing, Huntington's disease, Alzheimer's disease, Parkinson's disease, amyotrophic lateral sclerosis, cerebral hypoxia, stroke, and concussion.

As used herein the terms “treating” and “preventing” and grammatical equivalents refer to any and all uses which remedy a disease or disorder, prevent the establishment of a disease or disorder, or otherwise prevent, hinder, retard, or reverse the progression of a disease or disorder or any one or more symptoms thereof. Thus the term “treating” is to be considered in its broadest context. For example, treatment does not necessarily imply that a patient is treated until total recovery.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1—Preparation of a Complex Comprising Silica and Probucol

Probucol was dissolved in ethanol at a ratio of 1:10 by weight and sonicated for between 5 to 10 minutes to improve solubility of the probucol. An amount of silica was added to the solution of probucol in ethanol and stirred for about 30 minutes at room temperature. The solvent was removed via rotary evaporation under reduced pressure with stirring (100 rpm) at a temperature of 40° C. The pressure was decreased according to the following program: 800 mbar for 10 minutes, 100 mbar for 20 minutes and 1 mbar for half an hour. Different pressure ramps influence the formation of crystalline probucol on the outside of the silica particles. The ramp rate described in this example minimises the formation of crystalline probucol and maximizes the amount of probucol loaded within the pores of the mesoporous silica.

### Example 2—Physical and Structural Characterization of Calcined Silica

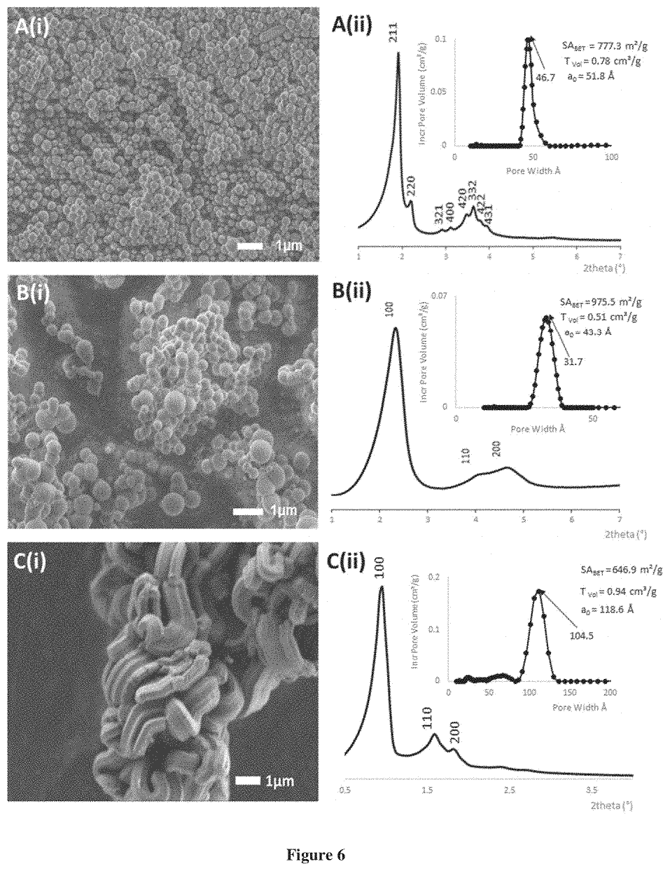

Samples of calcined silica (AMS-6, MCM-41 and SBA-15) were analysed by scanning electron microscopy and X-ray diffraction. Images are shown in FIG. 6.

### Example 3—Thermogravimetric Analysis of Mesoporous Silica Loaded with Probucol

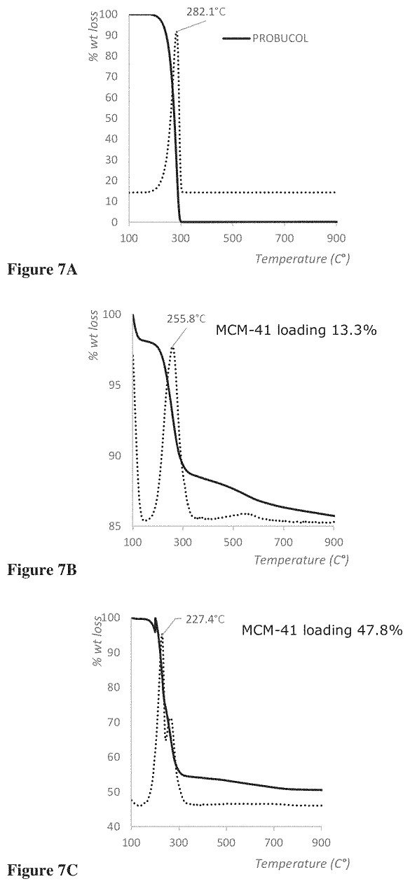

Samples of mesoporous silica (AMS-6, SBA-15 and MCM-41) loaded with probucol according to the procedure as described in Example 1 were subjected to thermogravimetric analysis. The TGA curves (percentage weight loss plotted as a function of temperature) are shown in FIG. 7.

### Example 4—Nitrogen Adsorption-Desorption Isotherm Curves for Calcined and Probucol Loaded Samples

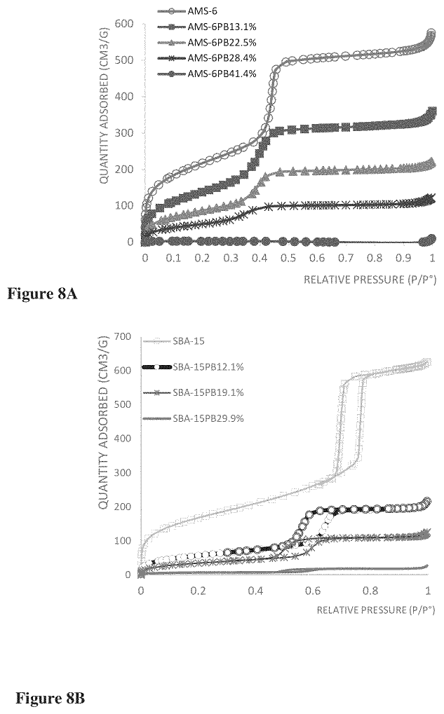

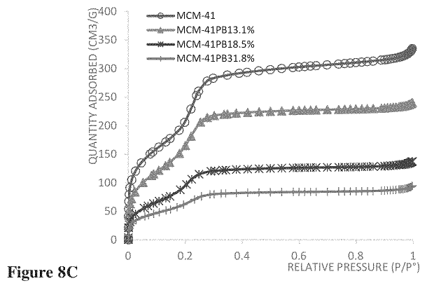

Nitrogen adsorption-desorption isotherm curves were obtained for calcined samples and silica samples (AMS-6, SBA-15 and MCM-41) loaded with probucol. The isotherms (quantity of nitrogen adsorbed plotted as a function of relative pressure of nitrogen) are shown in FIG. 8.

### Example 5—Dissolution of Mesoporous Silica Loaded with Probucol

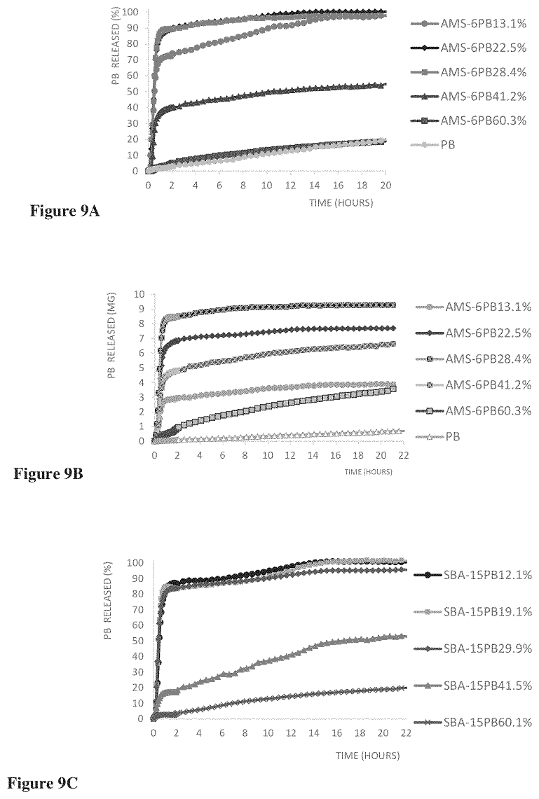

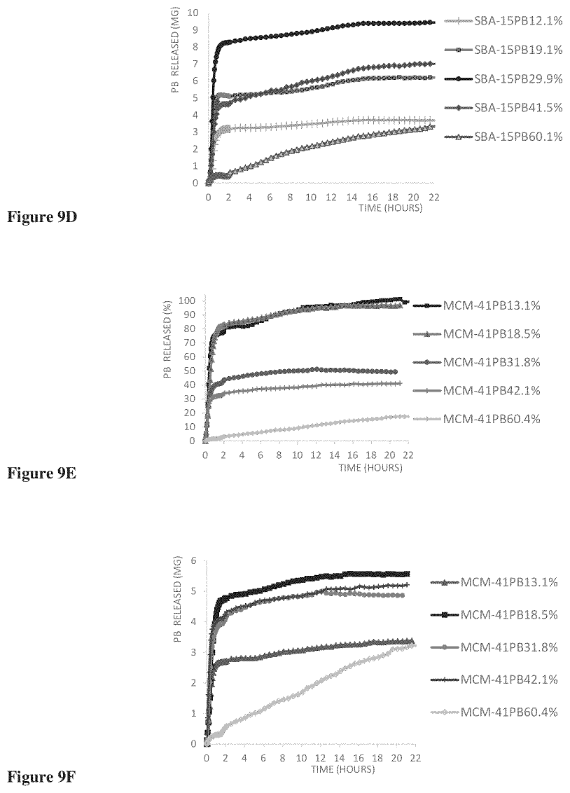

Samples of silica (AMS-6, SBA-15 and MCM-41) were loaded with probucol according to the procedure as described herein and the dissolution of the samples under sink conditions in simulated intestinal fluid was observed. The dissolution curves (quantity of probucol released plotted as a function of time) are shown in FIG. 9.

### Example 6—Dissolution of Capsules Containing Mesoporous Silica Loaded with Probucol

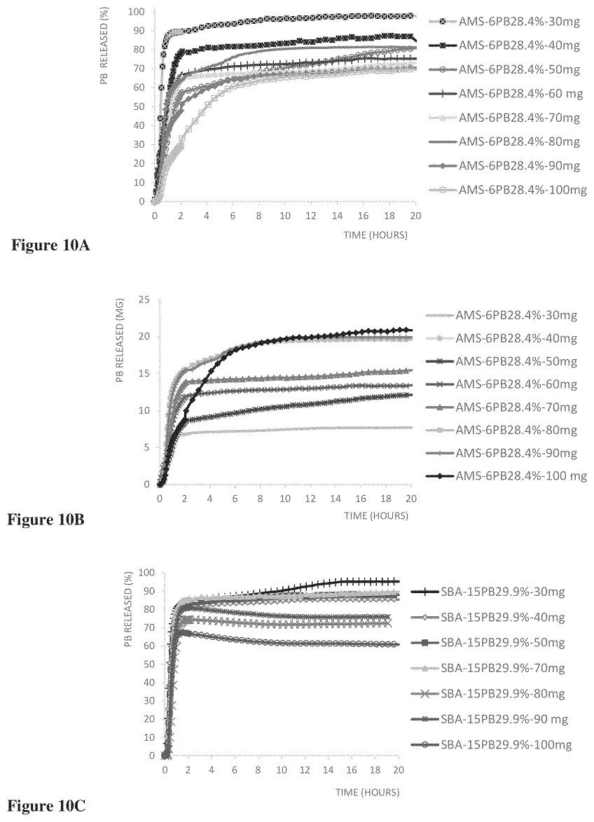

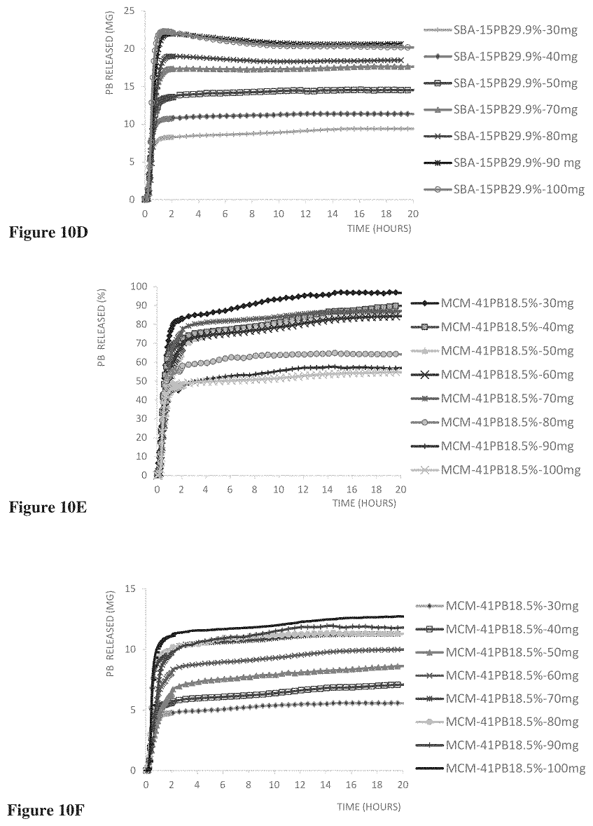

Samples of silica (AMS-6, SBA-15 and MCM-41) were loaded with probucol according to the procedure as described herein and the resulting complex was loaded into capsules at a weight from 30 mg to 100 mg. Dissolution of the capsules under sink conditions in simulated intestinal fluid was observed. The dissolution curves (quantity of probucol released plotted as a function of time) are shown in FIG. 10.

### Example 7—Fitted Drug Release Kinetic Parameters

The drug release kinetic parameters of mesoporous silica (AMS-6, SBA-15 and MCM-41) loaded with probucol (amounts given as a percentage by weight) according to the procedure as described in Example 1, were obtained from the dissolution experiments in simulated intestinal fluid as described above. The parameters were obtained using both Higuchi (H) and Korsmeyer-Peppas (KP) models (see Table 1).

TABLE 1

| | | H | | T_{1/2} | KP | | |
|---|---|---|---|---|---|---|---|
| PB | wt % | t_{lag}R² | K_{H} | (min) | t_{lag}R² | K_{KP} | n |
| 3D-Meso1 | 13.1 | 0.98 | 95.9 | 27.1 | 0.98 | 1.6 | 0.55 |
| | 22.5 | 0.96 | 112.5 | 26.7 | 0.98 | 0.4 | 1.5 |
| | 28.4 | 0.97 | 123.8 | 26.8 | 0.99 | 0.5 | 1.4 |
| | 41.2 | 0.97 | 49.3 | 635.1 | 0.96 | 1.6 | 0.4 |
| | 51.9 | 0.97 | 14.7 | — | 0.99 | 0.2 | 0.13 |
| | 60.3 | 0.98 | 4.1 | — | 0.99 | 0.8 | 0.51 |
| 2D-Meso1 | 13.1 | 0.97 | 80.2 | 26.7 | 0.95 | 1.2 | 0.6 |
| | 18.5 | 0.97 | 71.5 | 50.1 | 0.97 | 0.61 | 1.2 |
| | 31.8 | 0.97 | 45.5 | 605.1 | 0.98 | 1.6 | 0.3 |
| | 42.1 | 0.94 | 35.7 | — | 0.95 | 0.9 | 0.6 |
| | 47.8 | 0.98 | 13.3 | — | 0.97 | 1.1 | 0.3 |
| | 60.4 | 0.86 | 2.1 | — | 0.97 | 0.3 | 0.7 |
| 2D-Meso2 | 12.1 | 0.97 | 32.6 | 155.5 | 0.97 | 1.39 | 0.27 |
| | 19.1 | 0.98 | 35.6 | 335.1 | 0.97 | 0.69 | 1.03 |
| | 29.9 | 0.95 | 29.2 | 575.5 | 0.92 | 0.74 | 0.76 |
| | 41.5 | 0.95 | 17.15 | 1655.5 | 0.93 | 0.64 | 0.79 |
| | 49.5 | 0.96 | 9.23 | — | 0.96 | 0.95 | 0.24 |
| | 56.4 | 0.91 | 2.77 | — | 0.91 | 0.44 | 0.16 |

### Example 8—Pharmacokinetic Parameters in Sprague Dawley Rats

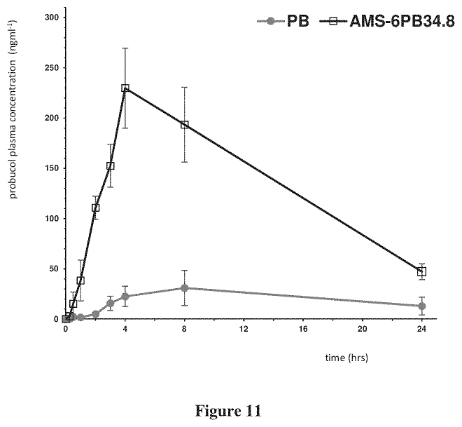

The in vivo pharmacokinetic parameters of mesoporous silica (AMS-6) loaded with probucol (34.8 wt %) prepared according to the procedure as described in Example 1, were obtained after oral gavage to male Sprague Dawley rats at a dose of 10 mg/kg. Crystalline probucol (PB) was administered in comparison. The mean plasma drug concentration-time curves and the key pharmacokinetic parameters are shown in FIG. 11 and Table 2. From Table 2, it can be seen that the time required to reach maximum concentration of probucol in vivo when mesoporous silica loaded with probucol is administered is approximately half that required for crystalline probucol. Further, the maximum concentration of probucol after administration of silica loaded with probucol is approximately 10 times higher than when crystalline probucol is administered.

TABLE 2

| Parameters | PB | AMS-6 PB34.8 wt % |
|---|---|---|
| tₘₐₓ (h) | 8 | 4 |
| Cₘₐₓ (ng · ml⁻¹) | 30.89 | 229.71 |
| AUC₀₋₂₄ (ng · h · ml⁻¹) | 492.27 | 3182.95 |
| AUC_{0-∞} (ng · h · ml⁻¹) | 858.81 | 3759.7 |
| t_{1/2} (h) | 19.64 | 8.48 |
| Tₗₐₛₜ (h) | 24 | 24 |

### Example 9—Reduction in Hydrogen Peroxide-Induced Cellular Oxidative Stress by Probucol

The antioxidant property of probucol loaded into AMS-6 mesoporous silica at 30 wt % was compared to vitamin C, and crystalline probucol at 100 μM (FIG. 5). The human cerebral microvascular endothelial cells (hCMEC/D3) line the microvasculature of the brain. The formation of tight junctions between hCMEC/D3 is a model of the blood brain barrier (BBB) in humans, which regulates exchanges between blood and the brain. Hydrogen peroxide is a ROS that is known to cause cellular oxidative stress. hCMEC/D3 cells were incubated with hydrogen peroxide at 1000 μM together with the test compounds. The percentage of cells with oxidative stress compared to the percentage of cells without oxidative stress were analysed through detection of the intracellular superoxide anion by using the MUSE® Oxidative Stress kit at incubation times of 2 to 48 hours (FIG. 5). Cells which are positive for the detection of ROS (% ROS positive) are considered as under oxidative stress, compared to cells which are not under oxidative stress (% ROS negative). The percentage of cells with oxidative stress was three times lower in AMS-6PB30% at 100 μM compared to crystalline probucol and vitamin C after an incubation time of 2 hours. The percentage of cells with oxidative stress was significantly lower in AMS-6PB30% at 100 μM compared to ascorbic acid and crystalline probucol at longer incubation times of between 4 to 48 hours.

### Example 10—Reduction of Cellular Oxidative Stress and Lipopolysaccharide-Induced Cyclooxygenase Enzyme Activity by Probucol

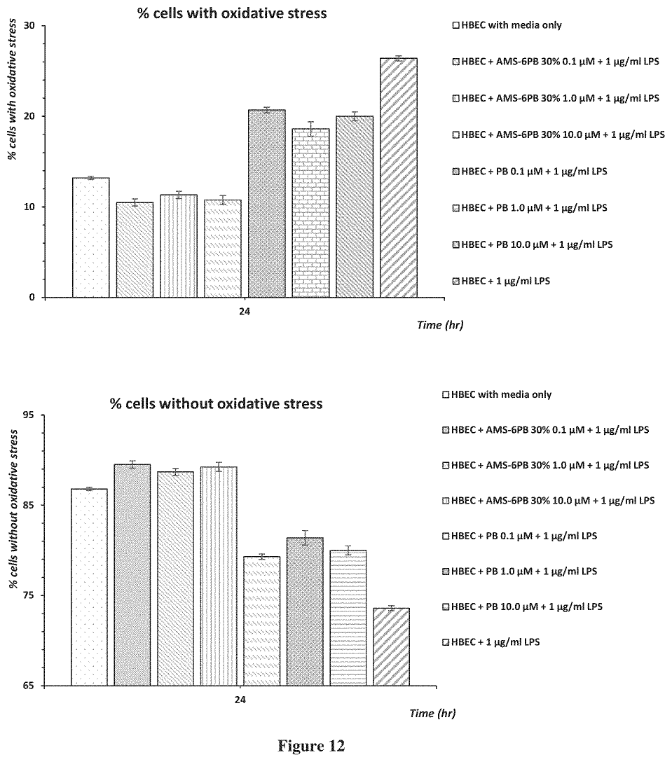

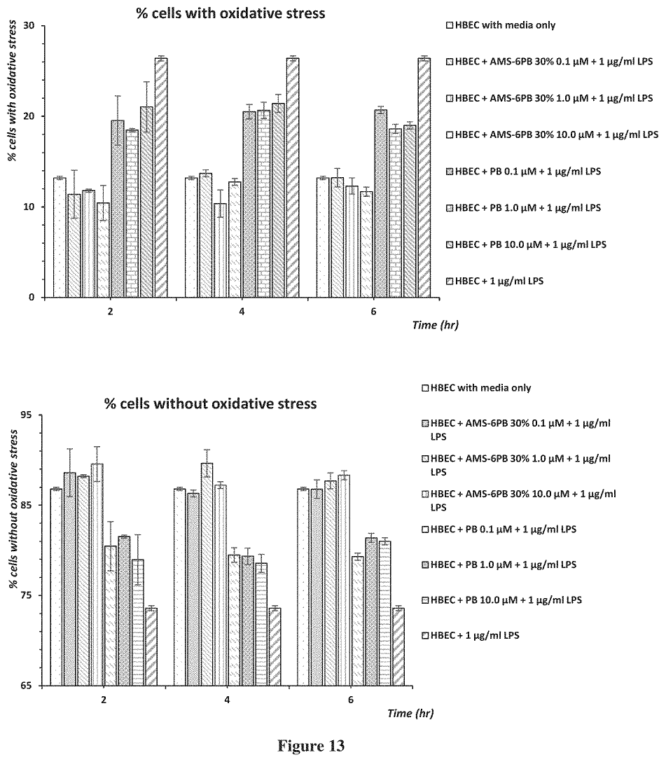

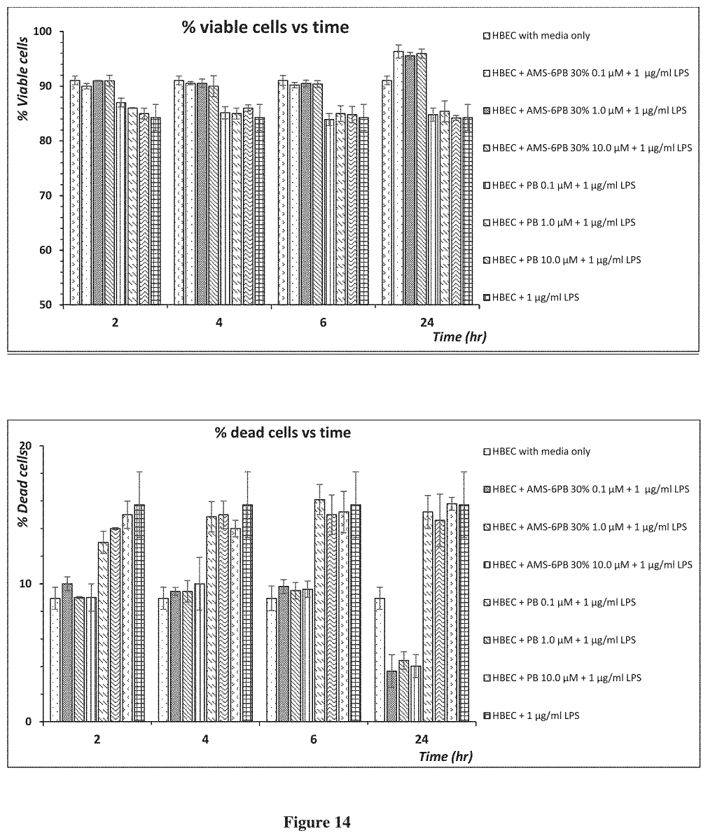

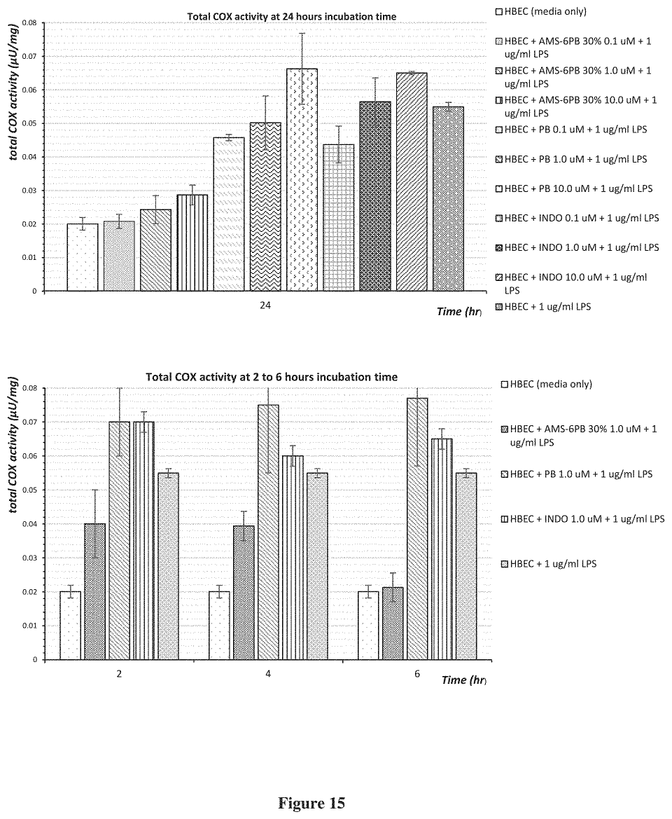

Tissue culture of primary human brain endothelial cells (HBEC) isolated from normal human brain tissue was used to further investigate the antioxidant and anti-inflammatory properties of probucol. Lipopolysaccharide (LPS) is an endotoxin found on the outer membrane of gram negative bacteria. LPS is known to cause cellular oxidative stress and inflammation by the activation of the toll like receptor 4 mediated production of superoxide anion from the mitochondria, and the COX enzyme mediated production of pro-inflammatory mediators respectively. The antioxidant property of probucol at different doses loaded into AMS-6 mesoporous silica at 30 wt % was compared to crystalline probucol by using the MUSE® Oxidative Stress kit. The test compound was added with 1 μg/ml LPS (lipopolysaccharide) and incubated for a period of 24 hours (see FIG. 12). The percentage of cells with oxidative stress was lower (by approximately 50%) at all doses tested after exposure to samples of probucol released from AMS-6, when compared to free crystalline probucol. The % cells with oxidative stress was significantly lower in probucol samples released from AMS-6 compared to free crystalline probucol at all doses tested. AMS-6 loaded with probucol shows a lower percentage of cells with oxidative stress compared to the negative control (HBEC with media only). In order to determine the free radical scavenging efficiency of probucol with or without loading in AMS-6, HBEC cells were incubated with 1 μg/ml LPS for a total period of 24 hours to induce cellular oxidative stress and inflammation. Test compounds were added for a total treatment duration of 2, 4 or 6 hours (see FIG. 13). The percentage of cells with oxidative stress was lower at all time points and doses with administration of samples of probucol loaded in AMS-6, when compared to the administration of crystalline probucol. Cellular viability was investigated by using the Muse® Count and Viability kit and shown in FIG. 14. After treatment with LPS, cellular viability was highest for those cell cultures incubated with AMS-6 loaded with probucol, regardless of the concentration used. After incubation for 24 hours with probucol loaded in AMS-6 and 1 μg/ml LPS, cellular viability was higher than the negative control. Levels of COX enzyme activity were measured using the COX Activity Assay kit (Abcam, ab204699), which measures both COX-1 and COX-2 enzyme activity. Total COX activity is lower in cells treated with probucol released from mesoporous silica AMS-6 at all concentration ranges (see FIG. 15) and lower than in cell lines treated with either crystalline probucol or the highly potent COX enzyme inhibitor and nonsteroidal anti-inflammatory drug indomethacin. Total COX enzyme activity after exposure to probucol released from AMS-6 at a dose of 1 μM was lower after incubation times of 2, 4 and 6 hours, when compared to crystalline probucol and indomethacin.

### Example 11—Dissolution of Capsules Containing Mesoporous Silica of Different Pore Size

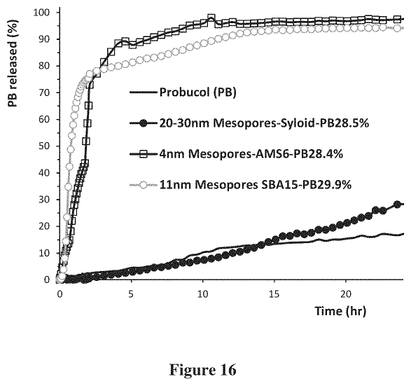

A comparison was made of solubility enhancement with capsules containing commercially mesoporous silica pharmaceutical excipient Syloid (with a pore size above 20 nm) and capsules containing low mesopore size capsules: AMS-6 (pore size 4 nm) and SBA-15 (pore size 11 nm), loaded with probucol at an approximate loading of 28.4% (AMS-6), 28.5% by weight. As shown in FIG. 16, a clear difference was observed in the dissolution profiles; the lower pore size mesoporous particles result in an enhancement of solubility at least 9 times higher than the larger pore size Syloid particles.

1. A complex comprising mesoporous silica and probucol, wherein at least a portion of the probucol is present within the pores of the silica.

2. The complex according to claim 1, wherein the probucol present within the pores of the silica is amorphous.

3. The complex according to claim 1, wherein the probucol is present in both an amorphous form and a crystalline form.

4. The complex according to claim 1, wherein the probucol present on the exterior surface of the silica is crystalline.

5. The complex according to claim 1, wherein the mesoporous silica has a pore size of between about 3.4 nm and about 30 nm.

6. The complex according to claim 1, wherein the mesoporous silica has a pore size in the range of about 12 nm to about 18 nm.

7. The complex according to claim 1, wherein the mesoporous silica has a pore size distribution between about 6 nm and about 20 nm.

8. The complex according to claim 1, wherein the mesoporous silica has a 2-dimensional pore structure.

9. The complex according to claim 1, wherein the mesoporous silica has a 3-dimensional pore structure.

10. A composition comprising the complex according to claim 1 and one or more pharmaceutically acceptable carriers, diluents or excipients.

11. A method for preparing the complex according to claim 1, the method comprising:
a. contacting mesoporous silica with a mixture of probucol in one or more solvents; and
b. removing the solvent.

12. The method according to claim 11, wherein the solvent is ethanol.

13. The method according to claim 11, wherein the solvent is removed by rotary evaporation under reduced pressure.

14. A method for increasing bioavailability of probucol in a subject, the method comprising administering to the subject the complex according to claim 1.

15. A method for lowering cholesterol in a subject, the method comprising administering to the subject the complex according to claim 1 or a composition thereof.

16. A method for treating a metabolic disease, cardiovascular disease, inflammatory disease, autoimmune disease, neurological disease, neurodegenerative disease, cancer, tumour, and/or other disorder associated with elevated cholesterol levels and/or oxidative stress, in a subject, the method comprising administering to the subject the complex according to claim 1 or a composition thereof.

17. A method for treating pain or inflammation in a subject, the method comprising administering to the subject the complex according to claim 1 or a composition thereof.

18. A method for inhibiting the activity of a cyclooxygenase enzyme in a subject, the method comprising administering to the subject the complex according to claim 1 or a composition thereof.

19. A product comprising a complex according to claim 1 for manufacture of a medicament for lowering cholesterol.

20. A product comprising a complex according to claim 1 for manufacture of a medicament for treating a metabolic disease, cardiovascular disease, inflammatory disease, autoimmune disease, neurological disease, neurodegenerative disease, cancer, tumour, and/or other disorder associated with elevated cholesterol levels and/or oxidative stress.

21. A product comprising a complex according to claim 1 for manufacture of a medicament for treating pain or inflammation.

22. A product comprising a complex according to claim 1 for manufacture of a medicament for inhibiting the activity of a cyclooxygenase enzyme.

23. The product according to claim 22, wherein the inhibition of cyclooxygenase activity is associated with quenching of intracellular superoxide.
